(19) United States
(12) Patent Application Publication
(10) Pub. No.: US 2019/0216769 A1
(43) Pub. Date: Jul. 18, 2019

(54) Topical Application of Delphinidin Compounds Treats Rosacea

(71) Applicant: HP Ingredients Corp., Bradenton, FL (US)

(72) Inventors: Juan Carlos BERTOGLIO, Valdivia (CL); Juan Eduardo CARRASCO-ZUBER, Valdivia (CL)

(73) Assignee: HP Ingredients Corp., Bradenton, FL (US)

(21) Appl. No.: 16/358,798

(22) Filed: Mar. 20, 2019

(51) Int. Cl. A61K 31/353; A61P 37/00; A61P 17/00; A61K 9/00; A61K 9/06

(52) U.S. Cl. CPC A61K 31/353; A61P 37/00; A61K 9/06; A61K 9/0014; A61P 17/00

(57) ABSTRACT

Topical application of Delphinidin twice per day treats rosacea and maintains normal healthy skin in patients with rosacea. Delphinidin applied topically increases and nourishes collagen and promote healthy skin.

### CROSS-REFERENCE TO RELATED APPLICATIONS

None.

### STATEMENT REGARDING FEDERALLY-SPONSORED RESEARCH

None.

### NAMES OF THE PARTIES TO A JOINT RESEARCH AGREEMENT

None.

### REFERENCE TO SEQUENCE LISTING

None.

### STATEMENT REGARDING PRIOR DISCLOSURES BY AN INVENTOR

None.

### BACKGROUND

Rosacea is a chronic skin disease. It affect the face: the cheeks, nose, eyes, chin and forehead. It is characterized by recurrent episodes of flushing, erythema (redness), papules (pimples), pustules and telangiectasia (permanent distended blood capillary vessels with a spidery appearance) (Elewski 2011; Korting 2009). There is to-date no satisfactory treatment.

We have found a treatment which is safe, easy to use and, as the appended photographs show, astonishingly effective. Our treatment can be used to treat rosacea and, alternatively, to maintain a normal healthy, attractive complexion.

Our invention involves the topical application of a delphinidin or similar compound. Our data show a significant reduction in skin flushing and redness, a benefit clearly superior to that of currently-known interventions.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1-38 are high-resolution color photographs of rosacea patients, illustrating their condition before treatment and after treatment for various intervals with the claimed composition.

### DETAILED DESCRIPTION

Rosacea primarily affects the face. It is accompanied by the physical discomfort of flushes, persistent erythema and the effects of eye lesions and inflammatory lesions. It can also lead to psychological problems over and above these physical symptoms. The disease can cause embarrassment, anxiety, low self-esteem and lack of confidence, and may even lead to depression, social anxiety disorder or body dysmorphic disorder (Abram 2009; Elewski 2011). If there is ocular involvement, it may result in varying and sometimes severe manifestations which may produce significant adverse clinical symptoms.

The severity of rosacea is related to the presence of papules and pustules. In contrast, patients with rosacea place greater quality of life emphasis on the erythema. There would appear to be poor consistency in assessments between individuals when detailed scoring scales are used (Bamford 2004). Moreover, several studies have demonstrated that objective clinical parameters of skin disease are often poorly correlated with quality of life. Thus, physicians tend to underestimate the impact of skin disease on an individual (Nicholson 2007). Rosacea has a significant negative impact on quality of life (Cresce 2014; Moustafa 2014).

Although there is no standard quantitative definition of the condition subtypes, rosacea is generally classified into four subtypes and one variant (Wilkin 2004). Subtype 1 is known as erythematotelangiectatic rosacea. Its clinical features include flushing and persistent central facial erythema (redness) with or without telangiectasia. Subtype 2 is known as papulopustular rosacea. It is characterized by persistent central facial erythema with transient, central face papules or pustules, or both. Subtype 3 is known as phymatous rosacea. It is characterized by thickening of the skin, and irregular surface nodules and enlargements. This may occur on the nose (rhinophyma), chin, forehead, cheeks or ears. Subtype 4 is known as ocular rosacea. It is characterized by ocular involvement, including inflammation of different parts of the eye and eyelid. It may be found in up to 58% of cases of rosacea, but it is frequently undiagnosed. The variant is known as granulomatous rosacea. It is non-inflammatory and characterized by hard, brown, yellow or red cutaneous papules, or nodules of uniform size. Progression from one subtype to another is possible. Patients may also express signs and symptoms of more than one subtype. Further, each individual characteristic or symptom may change over time from absent to severe (Tan 2013; Wilkin 2004).

Rosacea usually presents in the second or third decade of life. Up to 10% of the population can be affected (Berg 1989). The prevalence of rosacea varies from less than 1% to more than 20%, indicating a range which is most likely attributable to differences in the populations studied and the methodological approach used (Tan 2013b). It is reportedly more common in fair-skinned people of Celtic and northern European heritage (Korting 2009). Women appear to be more often affected than men (Culp 2009). However, a proportionately larger number of men develop phymatous changes (thickening skin, irregular surface nodularities, and enlargement), sometimes as a result of progression from subtype 1 or subtype 2 to subtype 3 (Wilkin 2004).

The pathophysiology of rosacea is very complex and the exact cause remains unclear. A number of hypotheses have been proposed. Both genetic and mostly environmental stimuli and triggers, for example heat, sunlight, stress, certain foodstuffs and Demodex mites stimulate an augmented innate immune response and neurovascular dis-regulation (Steinhoff 2013). In rosacea affected skin, elevated abnormal cathelicidin (an antimicrobial peptide) and elevated serine protease (kallikrein-5) induce increased LL-37, which results in inflammation, neurovascular effects and vascular changes (Del Rosso 2012). More recently mechanisms and components of rosacea pathophysiology have been categorised into (a) increase of Toll-like receptors on keratinocytes, (b) augmented innate immunity, (c) neurovascular dysregulation (d) vascular changes, (e) reactive oxygen species (ROS), (f) stratum corneum permeability barrier dysfunction, (g) ultraviolet (UV) radiation and (h) microbes, e.g. Demodex, Bacillus oleronius (Del Rosso 2012; Steinhoff 2011). The current hypothesis is that rosacea is an inflammatory disorder that may develop in individuals with rosacea-prone skin, initiated by several triggers (Steinhoff 2011). Possible triggers that have been investigated are gastrointestinal (digestive) tract diseases, infestation with Demodex folliculorum or Bacillus oleronius, epidermal barrier defect, and childhood stye (Bamford 2006; Elewski 2009).

A wide variety of treatments are available for this persistent (chronic) and recurring and often distressing disease. These include medications applied directly to the skin (topical), oral medications and light-based therapies (Elewski 2011; Powell 2005). Management strategies for people with rosacea can often be tailored to the specific subtype of rosacea (Powell 2005) but, because rosacea can have a significant impact on quality of life, these strategies should also be directed towards achieving improvements in general well-being (Elewski 2011). In certain individuals successful management of rosacea is possible through avoidance of some of the triggers, in particular those which cause flushing, that is, certain foods and beverages, sunlight and some types of cosmetics (Elewski 2011).

If a small number of papules and pustules are present, topical rather than systemic therapy is considered to be the first-line of treatment (Del Rosso 2013b; Elewski 2011). This includes a range of formulations of metronidazole, azelaic acid, clindamycin lotion, permethrin 5% cream, tretinoin cream, 10% sulphacetamide with sulphur (5%) and benzoyl peroxide alone or in combination with erythromycin or clindamycin (Culp 2009; Del Rosso 2013b; Elewski 2011). The Food and Drug Administration (FDA) and European Medicines Agency (EMA) approved topical ivermectin 1% for papulopustular rosacea. Brimonidine tartrate gel 5%, a topical selective α-adrenergic receptor agonist with vasoconstrictive activity, was recently approved for the treatment of persistent erythema in rosacea (Del Rosso 2013c).

If the skin lesions are more extensive, oral antibiotics such as tetracyclines or azithromycin are usually recommended (Culp 2009; Elewski 2011). Second-generation tetracyclines, for example doxycycline and minocycline, have been used more recently with reportedly fewer side effects (Sloan 2008). To reduce not only the side effects but also the risk of bacterial resistance, a novel approach has been advocated that involves the use of a sub-antimicrobial dosage of doxycycline, which separates the anti-inflammatory effect of doxycycline from its antimicrobial properties by limiting the plasma concentration to a range below the minimum inhibitory concentrations for susceptible bacteria (Baldwin 2010; Del Rosso 2007a). Not infrequently, oral treatment may be combined initially with topical treatment so that as the rosacea improves the systemic treatment can be discontinued and improvement can be maintained by continuation with the topical treatment alone (Elewski 2011).

The vascular manifestations of rosacea appear to respond fairly effectively to light-based therapies such as pulsed dye laser or intense pulsed light (Culp 2009; Tanghetti 2014). In the more severe or persistent cases of papulopustular and phymatous rosacea, oral 13-cis-retinoic acid (isotretinoin) therapy may be required. Isotretinoin has a well-recognised safety profile but should only be given to women under close supervision and should include an appropriate contraception strategy (Korting 2009; Nickle 2014). Traditional therapies for ocular rosacea include oral tetracyclines, eyelid hygiene and warm compresses (Vieira 2013). Topical metronidazole gel and fusidic acid gel are also reportedly successful (Vieira 2013). Rhinophyma may require surgical intervention, but low dose isotretinoin and laser therapy have also been used (Powell 2005; Tanghetti 2014).

Although a lack of understanding of the pathophysiology of rosacea continues to hamper therapeutic efforts (Elewski 2011), metronidazole and azelaic acid are generally considered as the first-line in topical medicaments. It is also now widely recognized that the therapeutic efficacy of both can be attributed to their anti-inflammatory and antioxidant effects (Elewski 2011; Feldman 2014). Ivermectin has demonstrated activity against Demodex in addition to possible inflammatory properties (Layton 2013). Brimonidine targets the α-adrenergic receptors in the smooth muscle sheath located around the vessel wall of the superficial blood vessels of the skin and coupled with its potent vasoconstrictive activity it can provide a reduction of facial erythema after application (Del Rosso 2013c). Tetracyclines down-regulate the production of inflammatory cytokines, inhibit matrix-metalloproteinases (MMP), and down-regulate the inflammatory response in papulopustular rosacea (Baldwin 2006; Del Rosso 2007a). Doxycycline has been shown to inhibit neutrophil activity and several pro-inflammatory reactions including those associated with phospholipase A2, endogenous nitric oxide and interleukin-6 (Baldwin 2006). Sub-antimicrobial doses of doxycycline can be important in minimizing the development of microbial resistance (Korting 2009). Isotretinoin has anti-inflammatory properties, it diminishes sebaceous gland size and number and can help retard or halt development of rhinophyma (Baldwin 2006; Uslu 2012).

Laser therapy is capable of reducing both erythema and telangiectasia (Shim 2013). The pulsed dye laser (PDL) with the 595 nm wavelength targets hemoglobin and delivers all of the administered energy in a wavelength that is actively taken up by the hemoglobin in blood vessels causing vessel destruction (Shim 2013). The 532 nm frequency-doubled, potassiumtitanyl-phosphate (KTP) and the neodymium-doped, yttrium-aluminum-garnet (Nd:YAG) laser can also deliver wavelengths which are readily absorbed by hemoglobin (Bernstein 2008). Intense pulsed light with a wavelength between 550 nm and 670 nm is readily absorbed by both melanin and oxy-hemoglobin, and has also been used in the treatment of telangiectasia (Nymann 2010).

Two separate treatments, metronidazole and azelaic acid, were effective and safe in reducing rosacea symptoms. Improvements tended to appear after three to six weeks. With metronidazole, very few people experienced mild itching, skin irritation and dry skin. For some, azelaic acid caused mild burning, stinging or irritation. More research is needed to conclude which of these two treatments is best. Ivermectin, a new treatment, was more effective than placebo and slightly more effective than metronidazole. Another newly registered treatment called brimonidine, especially for reducing redness, was shown to work up to 12 hours after being applied.

Antibiotics such as tetracycline, a low dose of doxycycline or a low dose of minocycline reduced the number of pimples and pustules. Low dose doxycycline (40 mg) was likely as effective as 100 mg, but with much fewer side effects of diarrhea and nausea. Azithromycin may be as effective as 100 mg doxycycline, but only one study addressed this treatment and better quality studies are needed to confirm this. A low dose of isotretinoin (0.3 mg/kg), a vitamin A-related drug, appeared to be slightly more effective than 50-100 mg doxycycline for treating pimples and pustules. However, extra precautions need to be taken regarding contraception in women of childbearing age as this drug is known to cause malformations in the fetus.

Laser therapy and intense pulsed light therapy were both effective for the treatment of telangiectasia, but the studies examining these treatments only reported limited data. For rosacea of the eyes or eyelids, or both (ocular rosacea), better quality studies are required on ocular rosacea, though cyclosporine 0.05% ophthalmic emulsion appeared to be more effective than artificial tears.

In contrast to the various prior art approaches, we tested the topical application of a standard skin cream with delphinidin derivatives. Commonly known as delphinidin, CAS 13270-61-1 has the UIPAC systemic name 2-(3,4,5-trihydroxyphenyl) chromenylium-3,5,7-triol and the following structure:

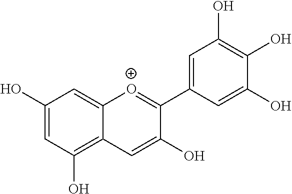 [SMILES: Oc1cc(O)c2cc(O)c(-c3cc(O)c(O)c(O)c3)[o+]c2c1]

It has several known analogs and derivatives. These include, for example delphinidin 3-O-sambubioside-5-O-glucoside, delphinidin 3,5-O-diglucoside, delphinidin-3-O-sambubioside and delphinidin-3-O-glucoside.

We found that the topical application of a variety of water-soluble Delphinidin compounds reduces the symptoms of redness and blushing in patients with rosacea. Further, the effect is surprisingly pronounced. Further, we found the effect consistent across different types of rosacea.

### Example 1

We obtained a topical skin cream base as is conventional in the art. We prefer a topical cream base made with aqua (deionized water), ethylhexyl palmitate, zemea (corn) propanediol, triethanolamine, Lavandula anguslifolia (lavender) oil, decyl glucoside, gluconolactone, sodium benzoate, acrylates/C10-30 alkyl acrylate crosspolymer, xanthan gum and sodium metabislufite. Alternatively, one can use a water-based cream using carboxyl methylcellulose in a concentration of about 1% as a gelling agent, with blackberry and plum extracts, and Gevuina avellanae (sea buckthorn) oil (a source of palmitoleic acid, an omega-7 fatty acid).

To this base, we added Delphinidin to make a topical cream with 1.0% (w/w) of Delphinidin. Alternatively, one could use as little as about 0.08% (w/w) or as much as >10% (w/w).

### Example 2

A 42-years-old female patient, suffering of papulopustular rosacea for the past years. During this period she received several classical treatments with no favorable response. The last course of treatment was corticosteroid topical cream, which worsened her condition by causing a severe facial infection where she required oral antibiotics.

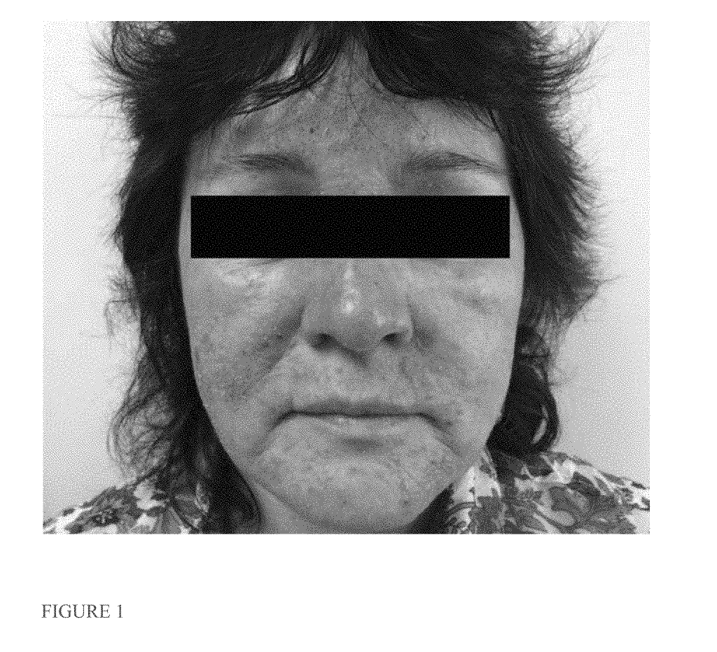

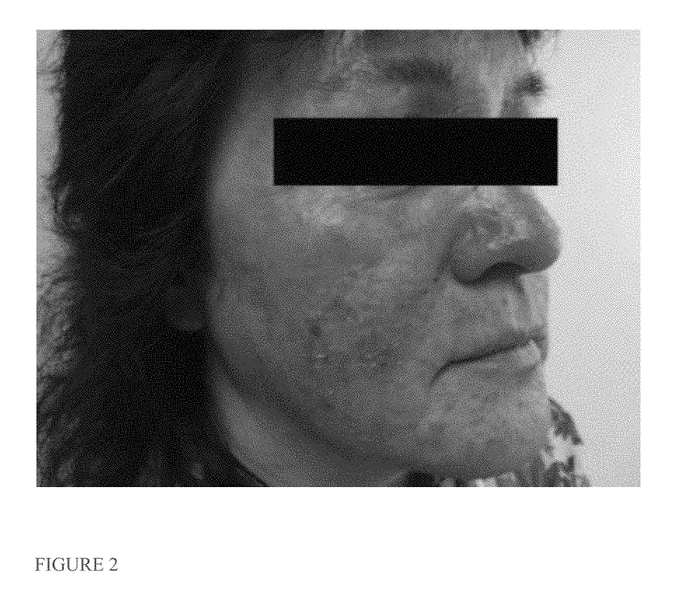

FIGS. 1 and 2 show color photographs of the patient's face at the beginning of the test. She was provided the cream of Example 1 and instructed to apply it to the face, twice a day, for six months. After two weeks of treatment, lesions began to fade away progressively, with a significant decrease in symptoms, i.e., reduction of redness, itching and blushing, without any observed adverse side effects. (data not shown)

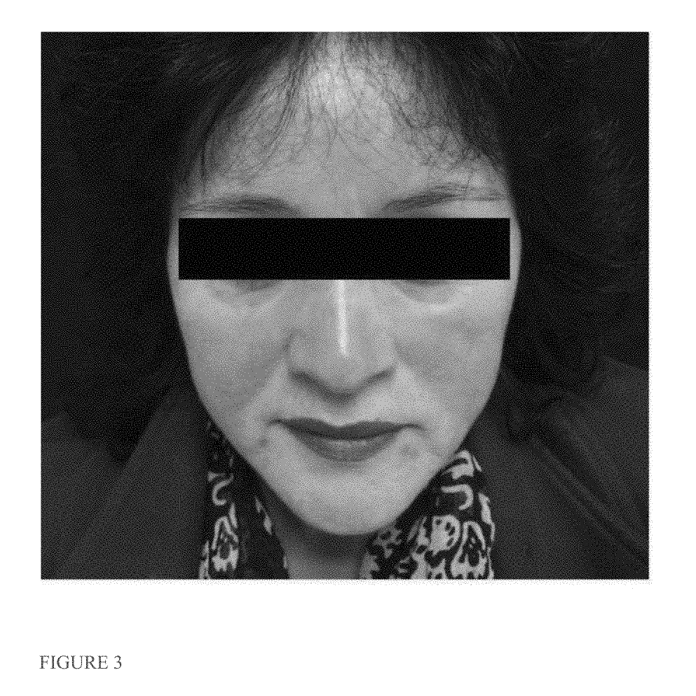

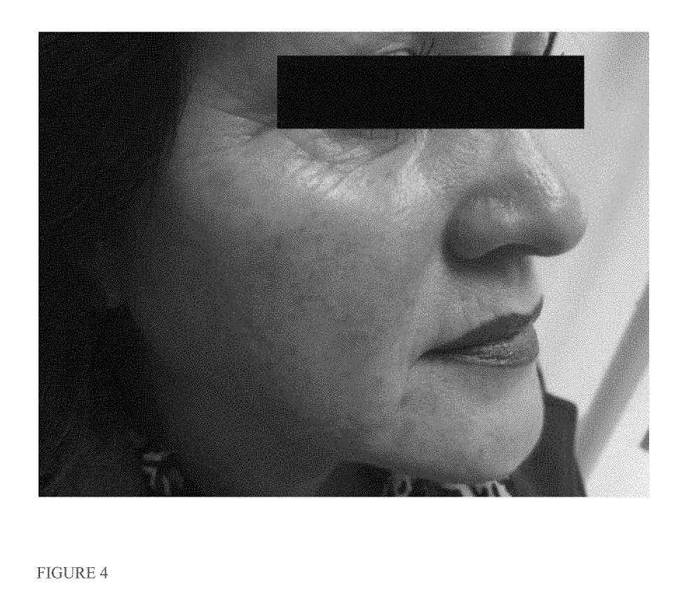

After two months of treatment, the patient had improved significantly. FIGS. 3 and 4 show color photographs of the patient's face after two months of treatment.

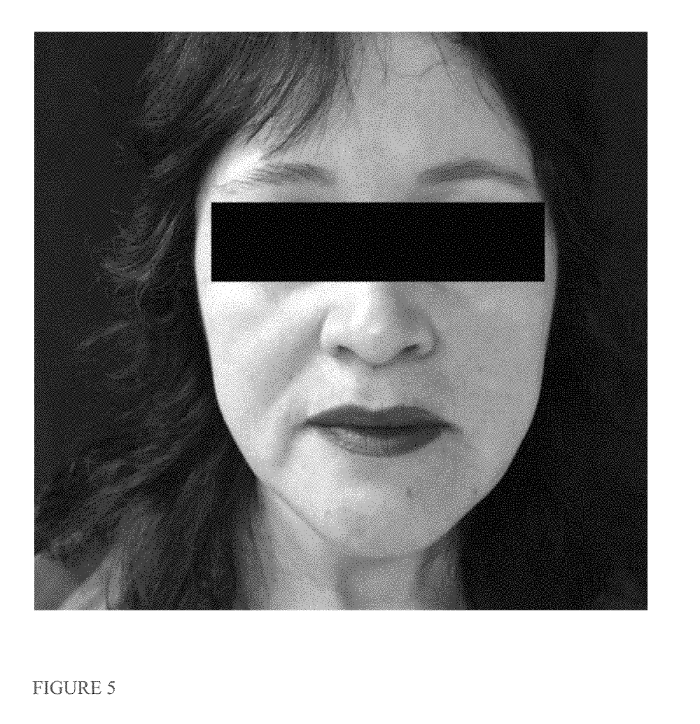

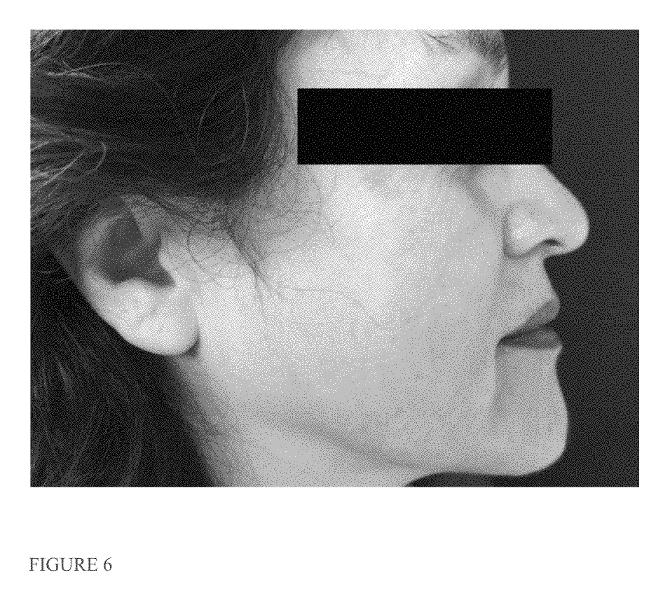

After six months of treatment, the patient exhibited full recovery, with complete absence of detectable symptoms and restoration of her skin to normal, healthy condition. FIGS. 5 and 6 show color photographs of the patient's face after six months of treatment.

### Example 3

Female patient, 38 years old, diagnosed with painful eritematotelangiectasic rosacea refractory to classical treatments for more than five years. Condition was exacerbated by hot weather and abrupt changes of environmental temperature.

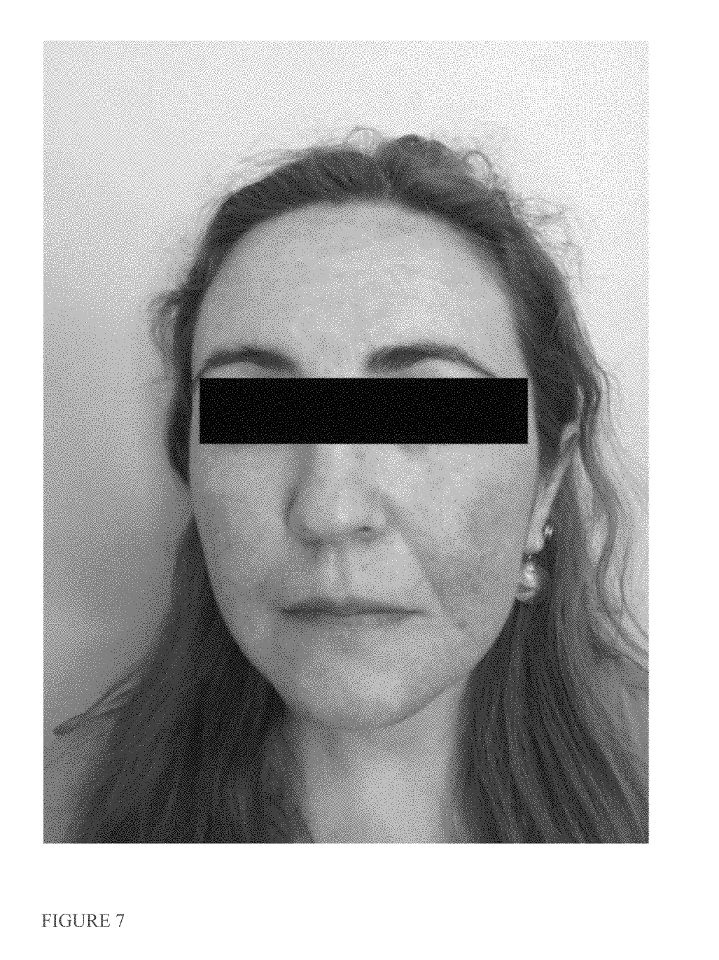

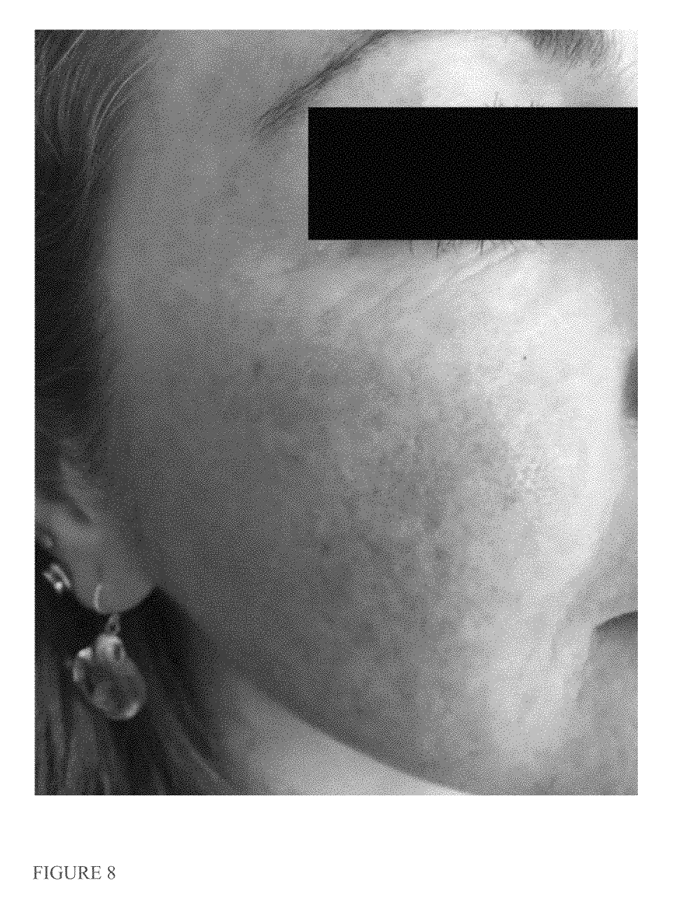

FIGS. 7 and 8 show color photographs of the patient's face at the beginning of the test. She was provided the cream of Example 1 and instructed to apply it to the face as monotherapy, twice a day, for two months.

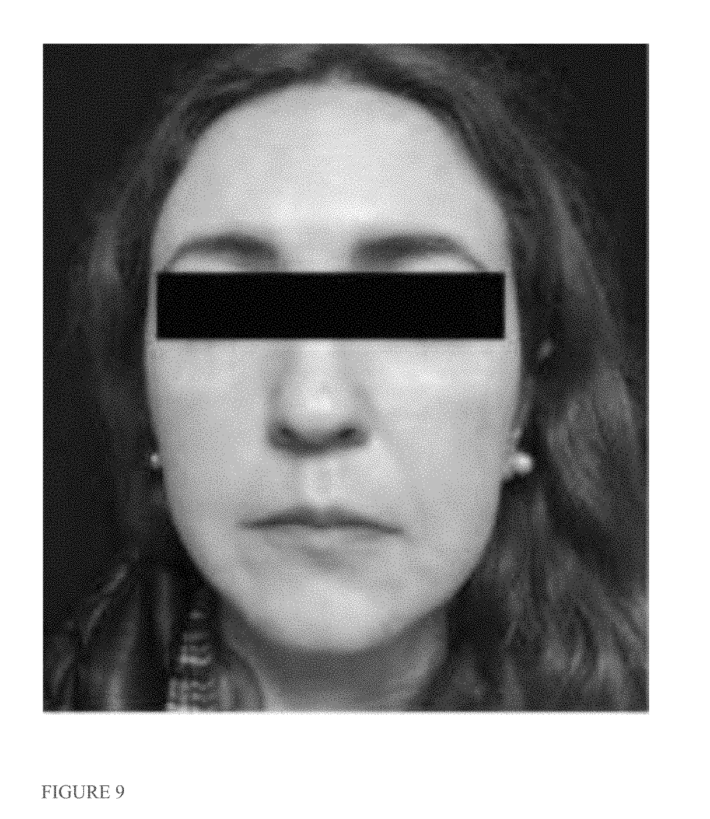

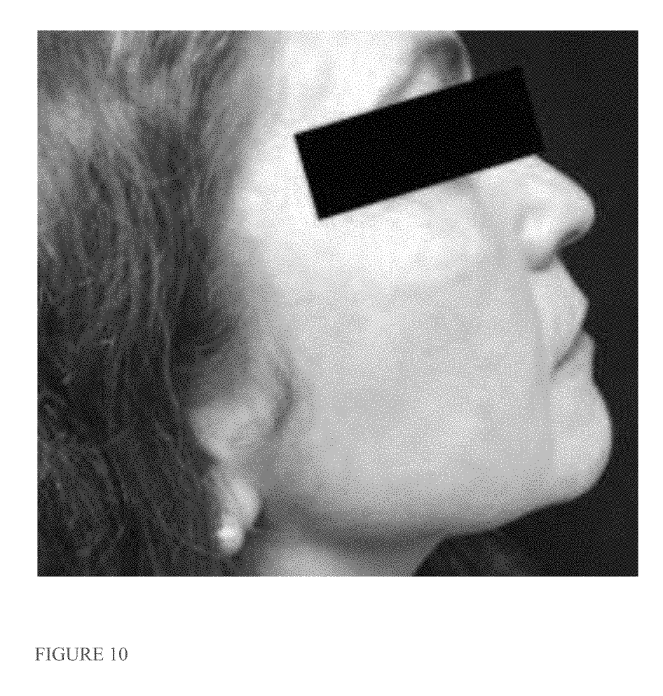

The patient experienced prompt pain relief (data not shown) and progression to full control of lesions, without any reported adverse side effects. FIGS. 9 and 10 show color photographs of the patient's face after 2 months of treatment.

### Example 4

A 35-years-old female with systemic lupus erythematosus and concomitant papulopustular painful rosacea of difficult management and refractory to classical treatments, exhibiting an overlap of telangiectasic facial lupus lesions and papular rosacea lesions.

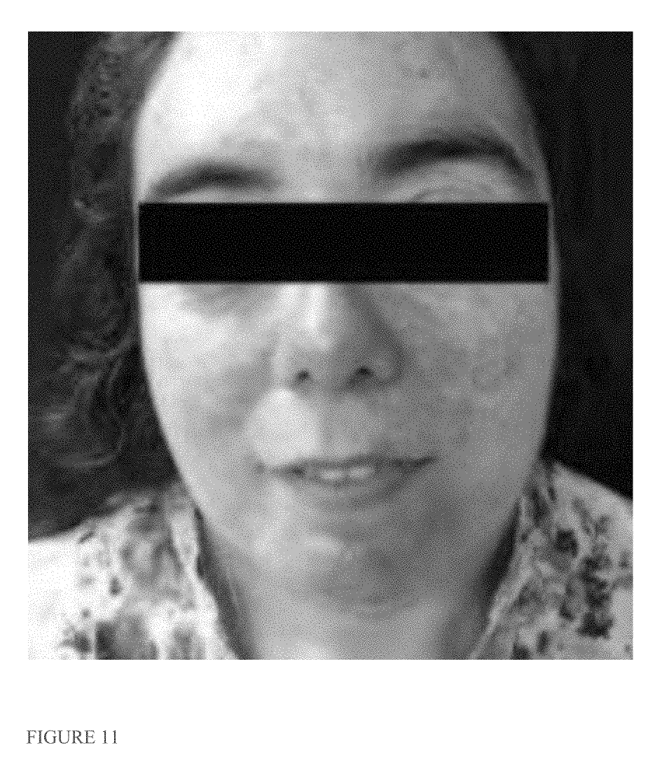

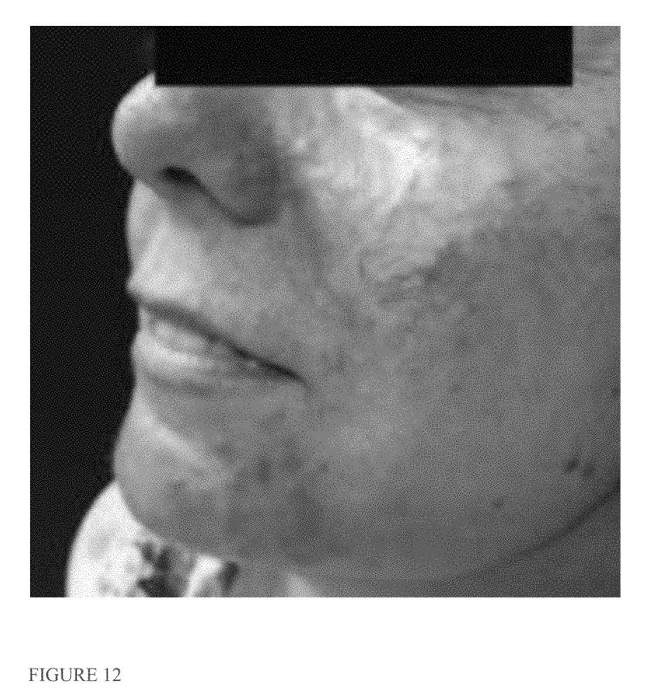

FIGS. 11 and 12 show color photographs of the patient's face at the beginning of the test. She was provided the cream of Example 1 and instructed to apply it to the face twice a day for two months.

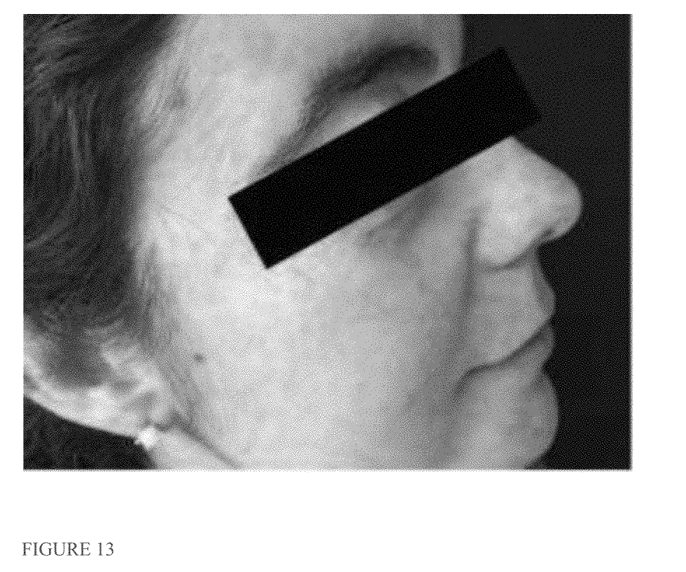

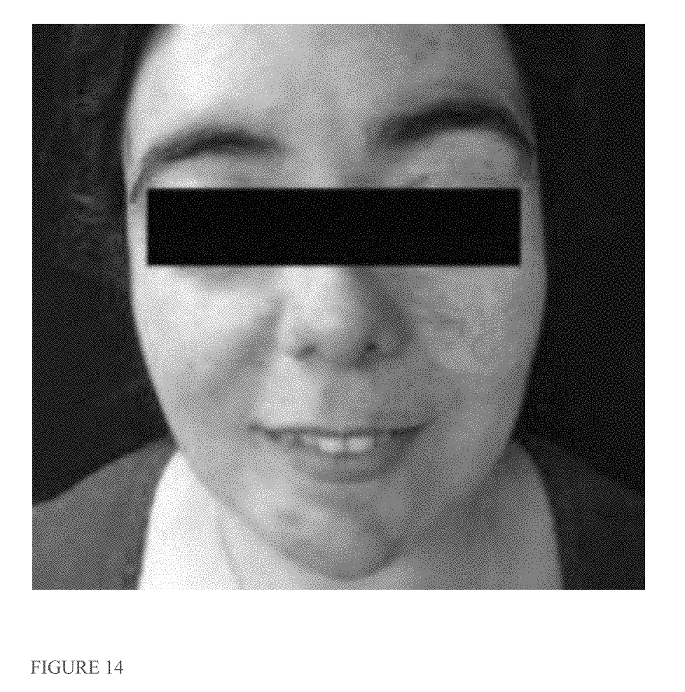

FIGS. 13 and 14 show color photographs of the patient's face after completing 2 months of treatment. Treatment for two months demonstrated an early and significant effect on connective tissue inflammation and on vascular proliferative activity. Treatment appeared to produce a selective effect, without any reported adverse side effects.

### Example 5

A 44 years old female with eritematotelangiectasic rosacea of difficult management, refractory to classical treatments for the past five years. This patient practiced gymnastic sports, complaining of severe painful blushing and intense redness reaction upon exercise warming up.

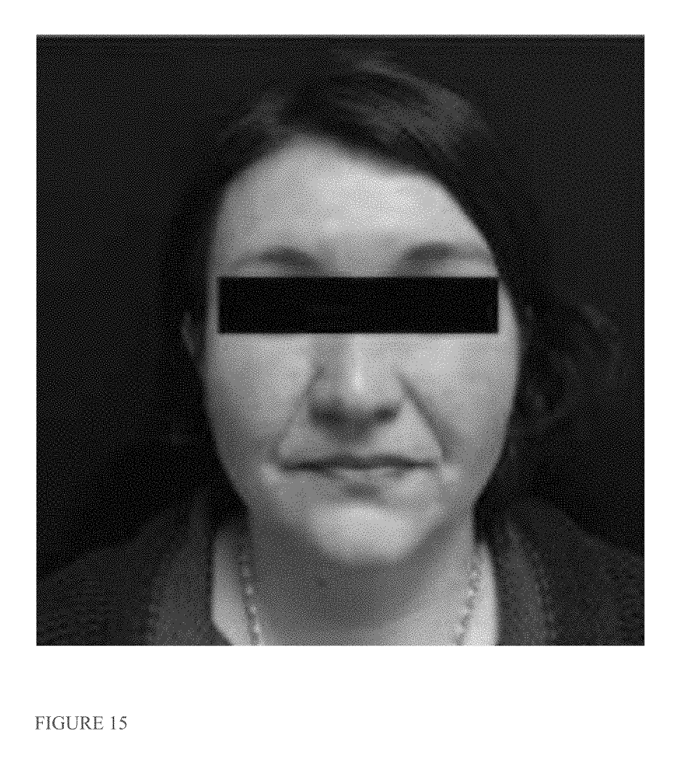

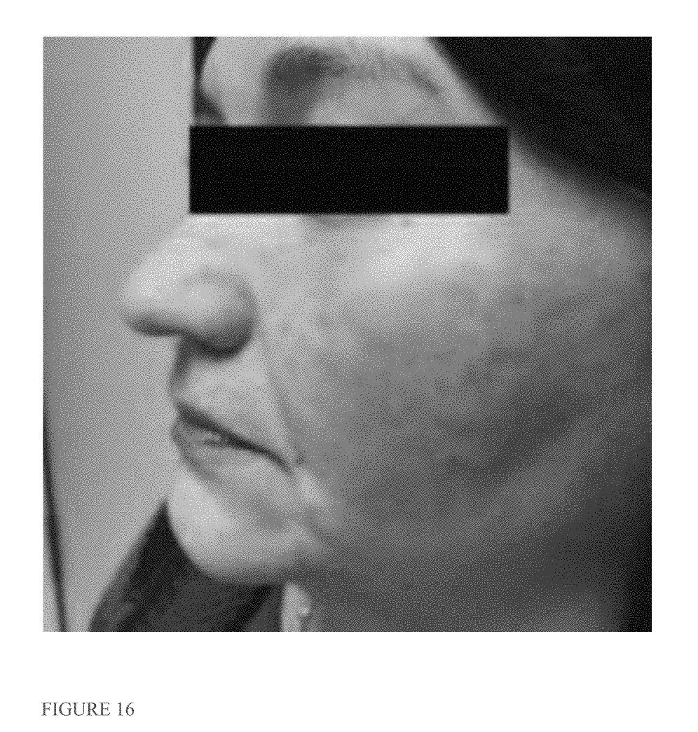

FIGS. 15 and 16 show color photographs of the patient's face at the beginning of the test. She was provided the cream of Example 1 and instructed to apply it to the face twice a day for two months.

After few days of treatment, the patient reported symptomatic relief to this triggering condition (data not shown).

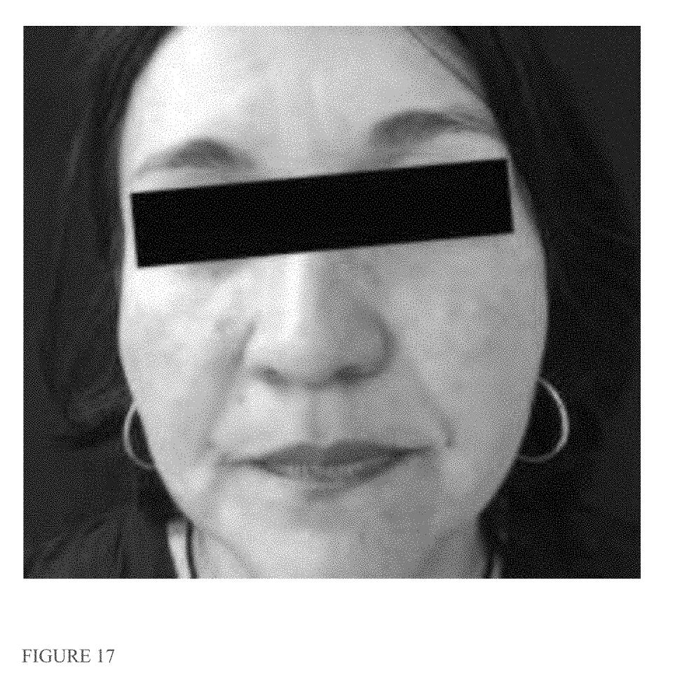

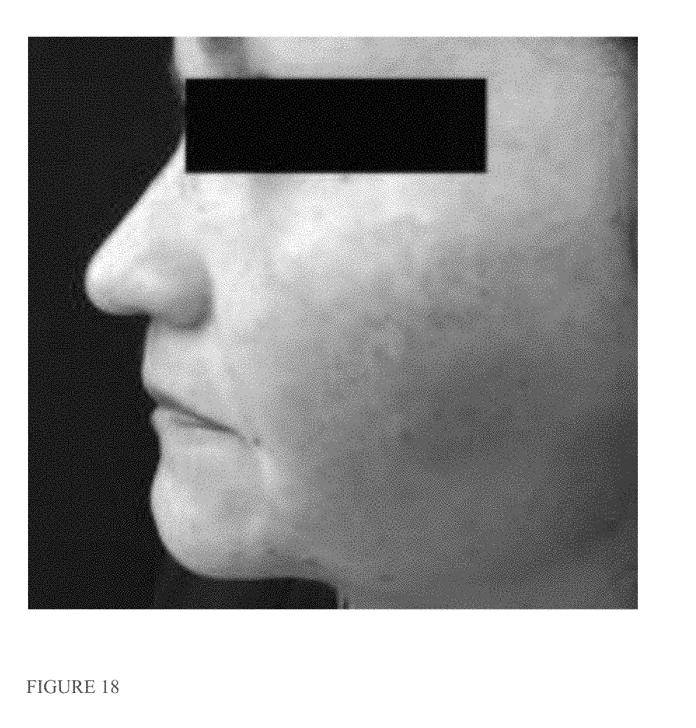

FIGS. 17 and 18 show color photographs of the patient's face after two months of treatment. These photographs show amelioration or elimination of rosacea symptoms and progression to recovery of normal healthy (non-inflammatory) basal skin structure. Treatment was well tolerated and the patient reported no adverse side effects.

### Example 6

A 57 year old female patient with eritematotelangiectasic painful rosacea, also with papular lesions localized mainly over the nose and untreated for the past twenty years. The patient lived at high altitude in a mountainous, often-snowy region. Her skin had thus been exposed to intense cold and sunlight, and significant outdoor temperature changes.

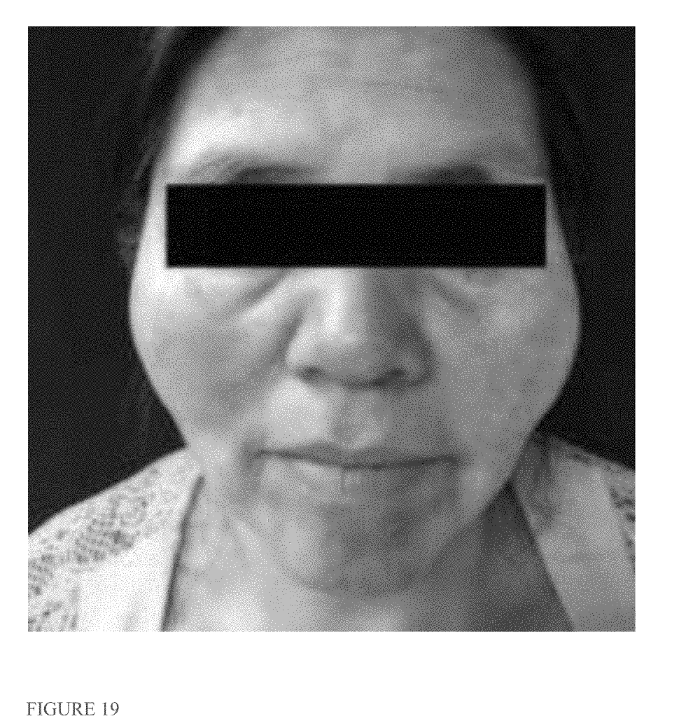

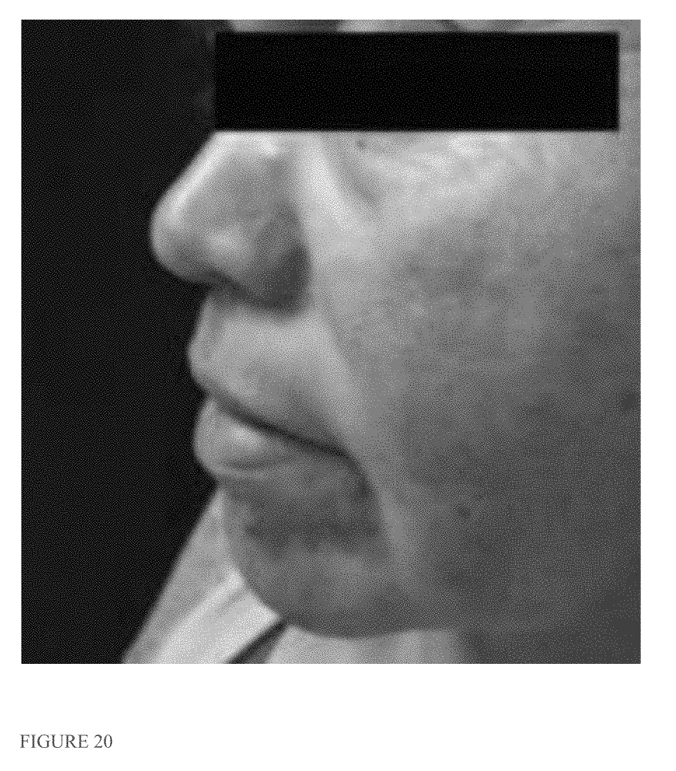

FIGS. 19 and 20 show color photographs of the patient's face at the beginning of the test. She was provided the cream of Example 1 and instructed to apply it to the face twice a day for two months.

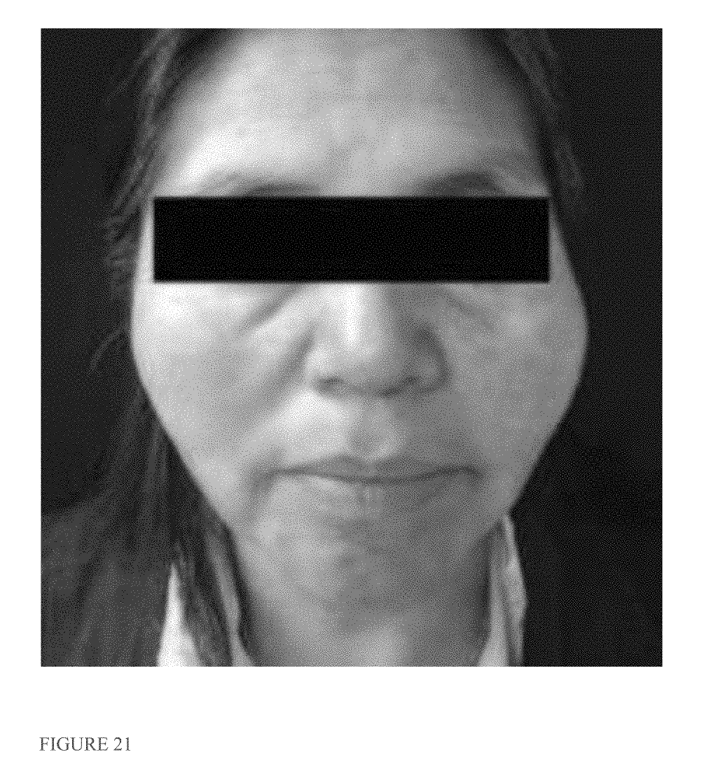

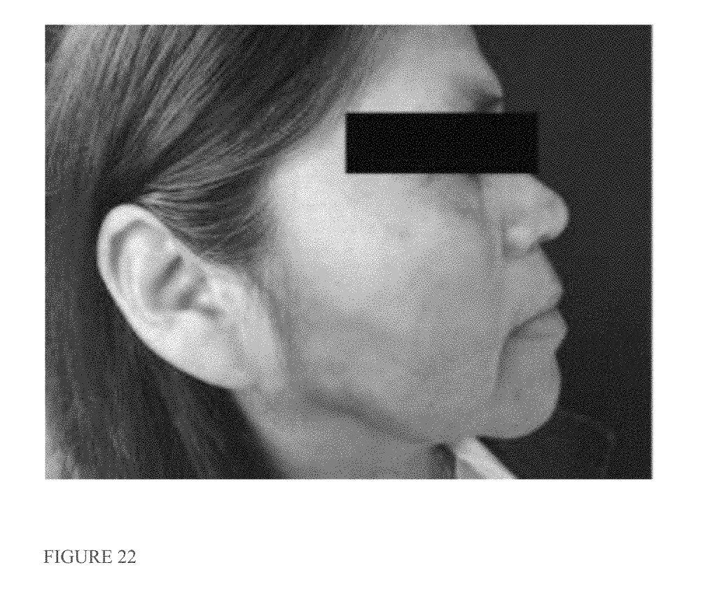

FIGS. 21 and 22 show color photographs of the patient's face after two months of treatment. These photographs confirm a significant improvement on nasal papular lesions redness and secondary erosions. The patient also reported amelioration of pain and itching (data not shown). No adverse reactions or tolerability problems were observed.

### Example 7

A 32 year old female with very symptomatic eritematotelangiectasic rosacea, unresponsive to classical treatments for the past sixteen years, suffering mainly pain over the cheeks. Her facial skin was exposed to unstable outdoor climatic heat, windy, rainy and cold conditions due to her occupation in the military.

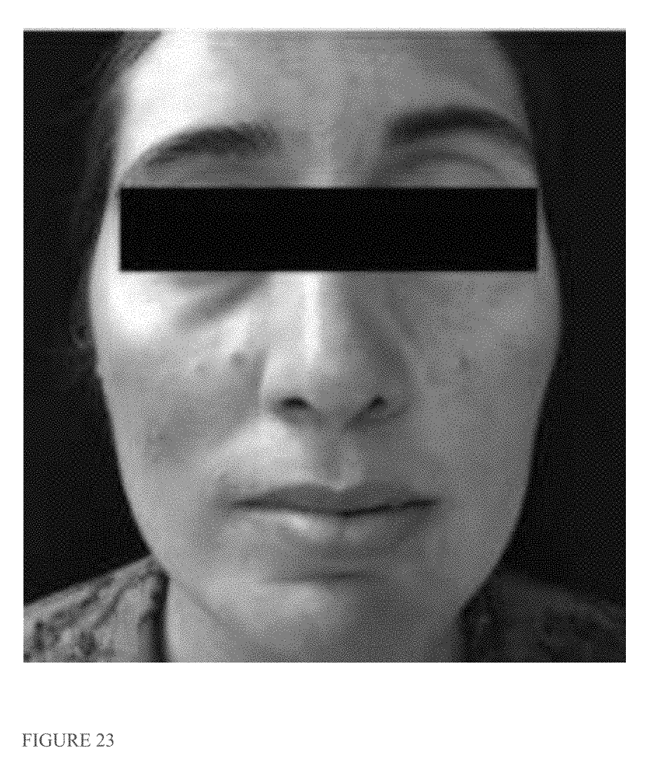

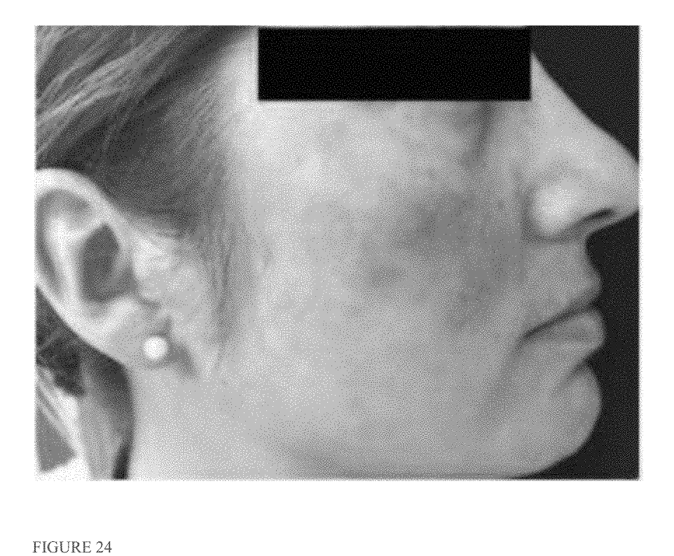

FIGS. 23 and 24 show color photographs of the patient's face at the beginning of the test. She was provided the cream of Example 1 and instructed to apply it to the face twice a day for two months.

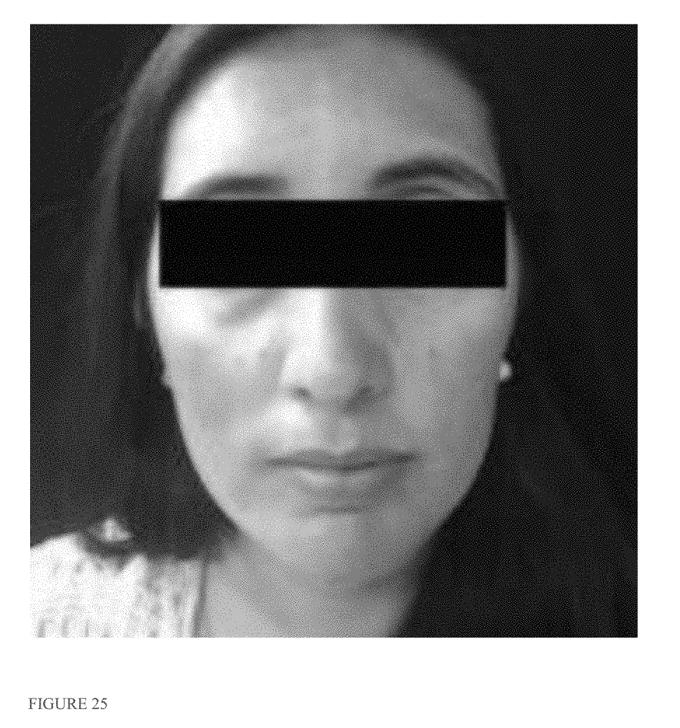

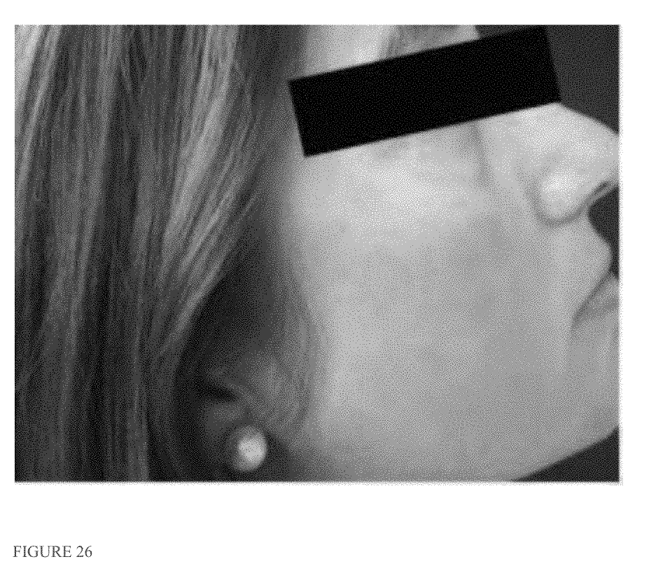

FIGS. 25 and 26 show color photographs of the patient's face after two months of treatment. These photographs demonstrate amelioration of inflammation and discoloration. In addition, the patient reported early and significant pain relief and recuperation of her previous facial skin softness. (data not shown) Treatment was well tolerated and the patient reported no adverse side effects.

### Example 8

A 25 year old female patient, university student, with eritematotelangiectasic rosacea and multiple failed classical treatments by different specialists, with severely deteriorated quality of life, due to intense painful and continuous blushing for the past four years.

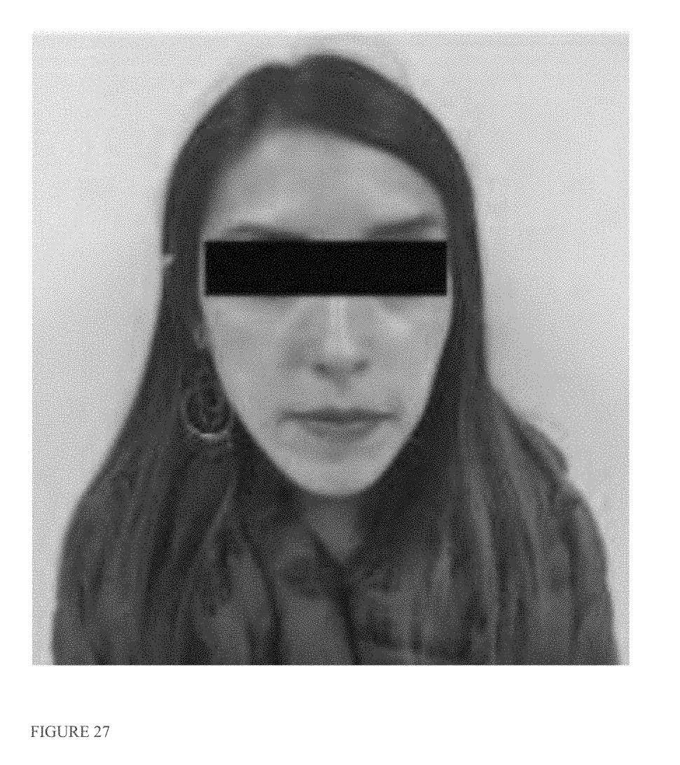

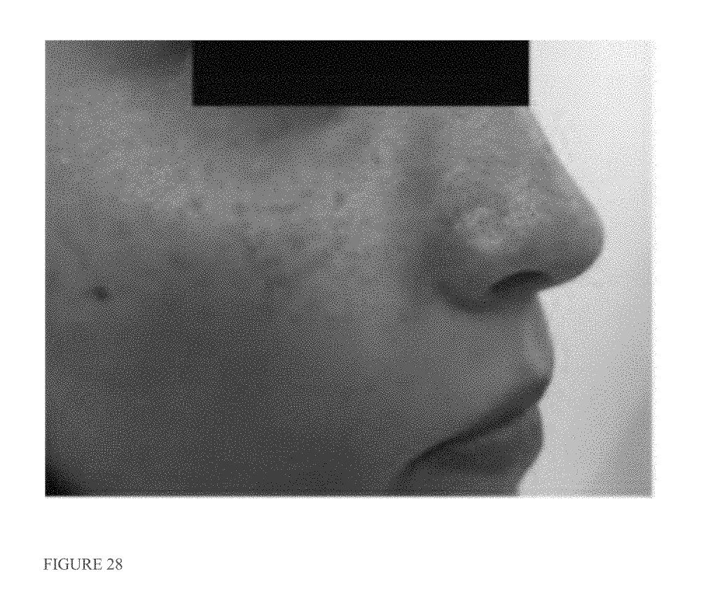

FIGS. 27 and 28 show color photographs of the patient's face at the beginning of the test. She was provided the cream of Example 1 and instructed to apply it to the face twice a day for two months.

After one week of treatment, the patient reported a significant reduction in skin pain, redness and burning sensation, with an almost-full recovery of her quality of life. (data not shown)

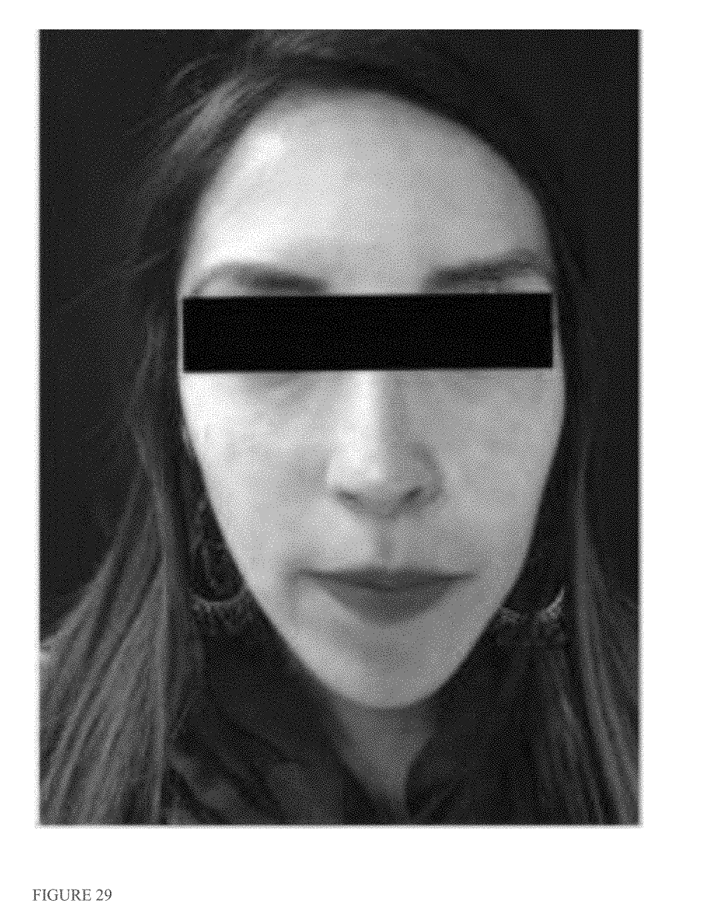

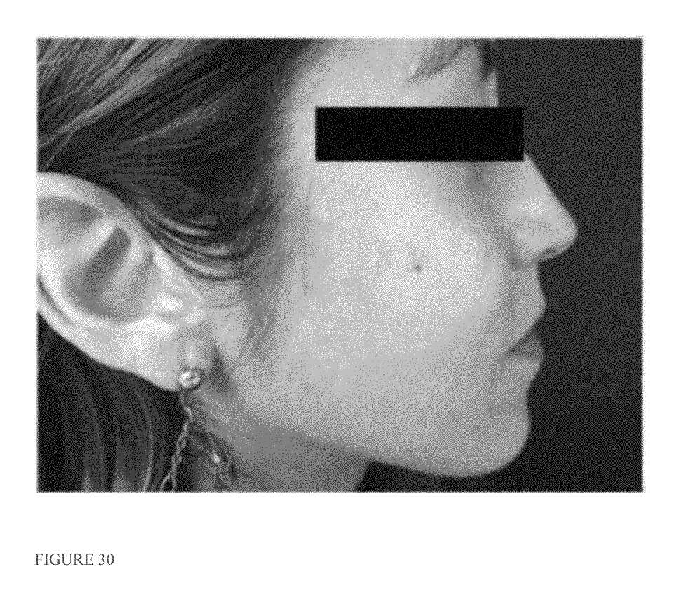

FIGS. 29 and 30 show color photographs of the patient's face after two months of treatment. These photographs demonstrate almost complete amelioration of inflammation and discoloration.

### Example 9

A 41 year old female patient, clerical administrative officer, with eritematotelangiectasic rosacea for the past ten years and never properly treated, complaining of intense flushing and continuous facial burning pain.

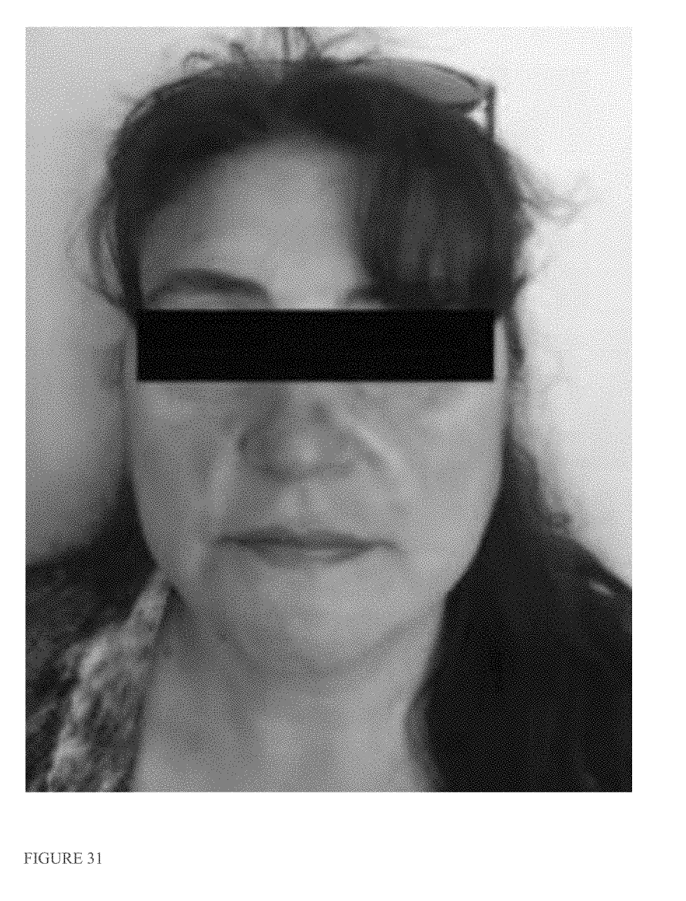

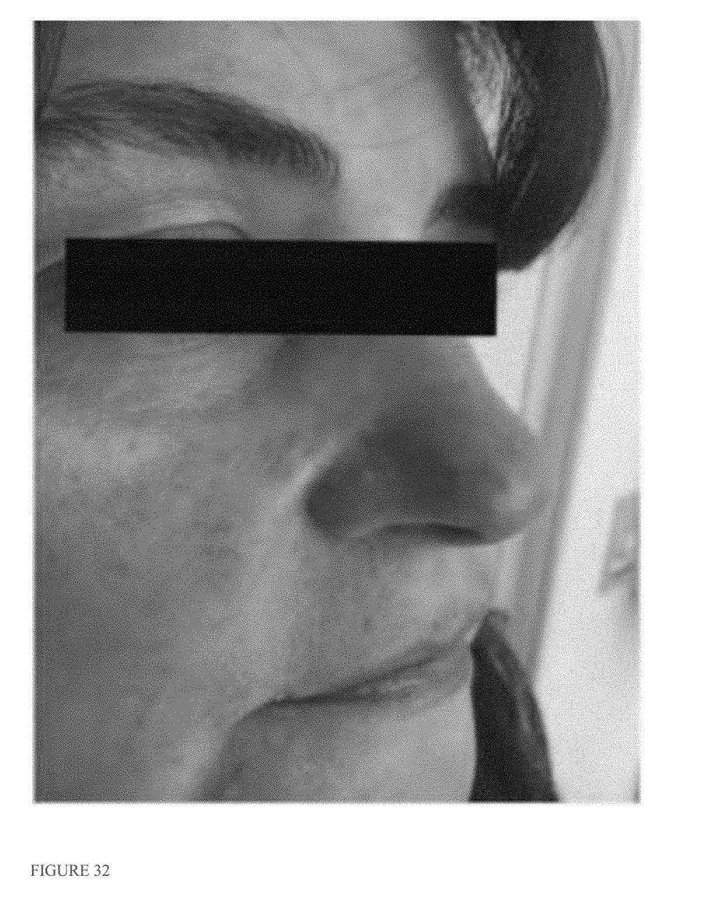

FIGS. 31 and 32 show color photographs of the patient's face at the beginning of the test. She was provided the cream of Example 1 and instructed to apply it to the face twice a day for two months.

Symptoms begun to progressively disappear at two weeks of delphinidin cream twice a day, with significant reduction of redness, itching and blushing. (data not shown)

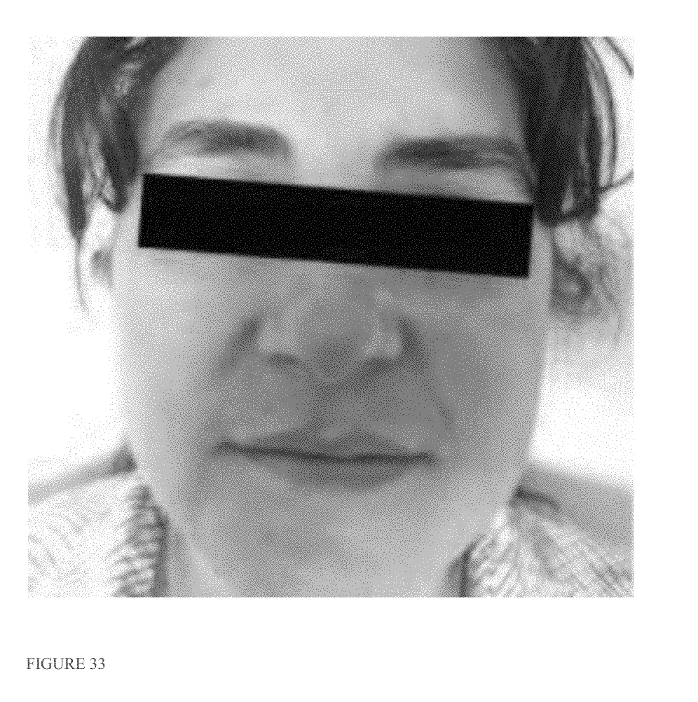

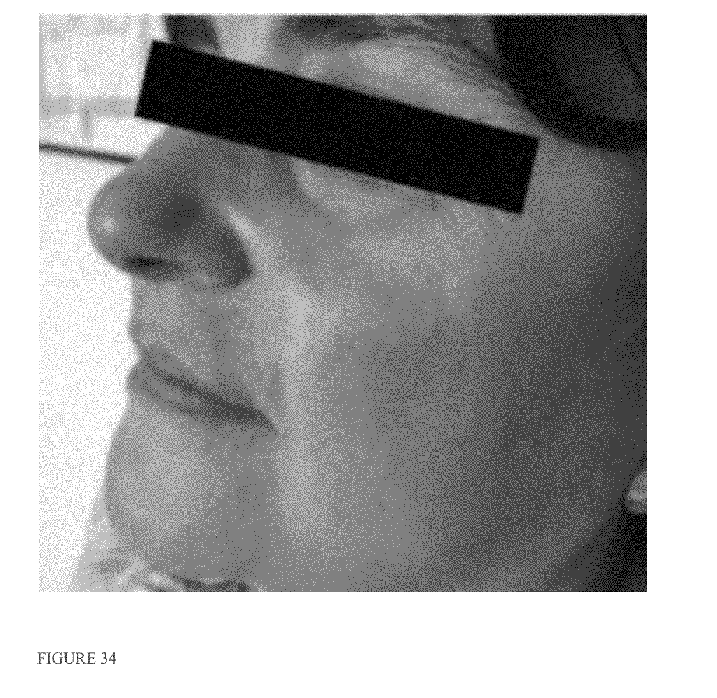

FIGS. 33 and 34 show color photographs of the patient's face after two months of treatment. These photographs demonstrate almost complete amelioration of inflammation and discoloration. No adverse side effects were observed.

### Example 10

A 28 years-old female patient, housewife, with relapsing papulopustular rosacea for the past three years after child delivery, with continuously increasing pustular and burning symptoms. In addition, this patient exhibited dry skin which worsened her inflammatory rosacea symptoms, particularly the itching sensation.

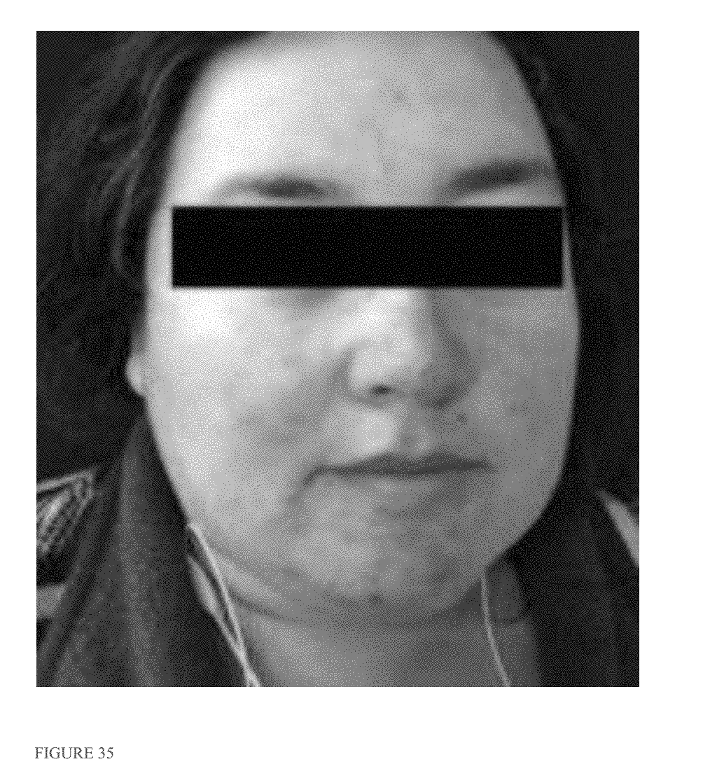

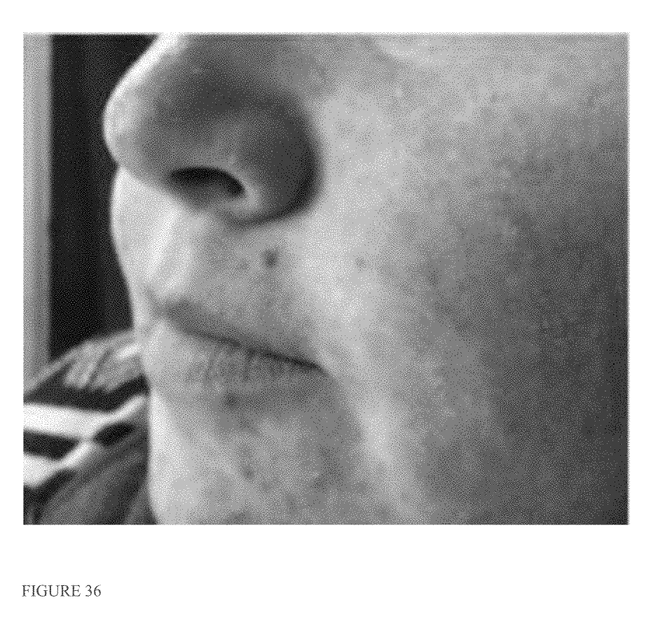

FIGS. 35 and 36 show color photographs of the patient's face at the beginning of the test. She was provided the cream of Example 1 and instructed to apply it to the face twice a day for two months.

After three to four weeks of treatment with delphinidin cream twice a day, the patient reported amelioration of her of dry skin condition, an absence of itching and a progressive fading away of redness and blushing. (data not shown).

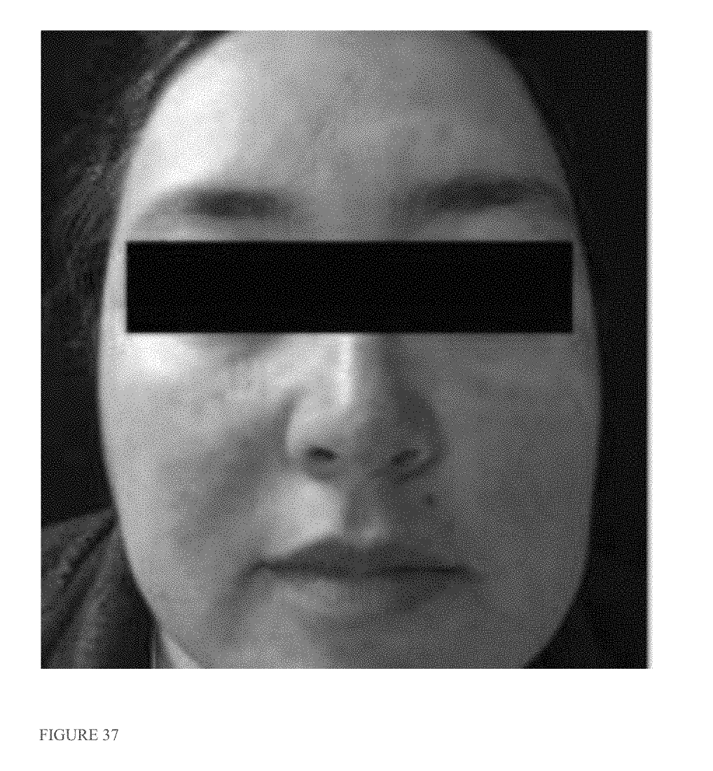

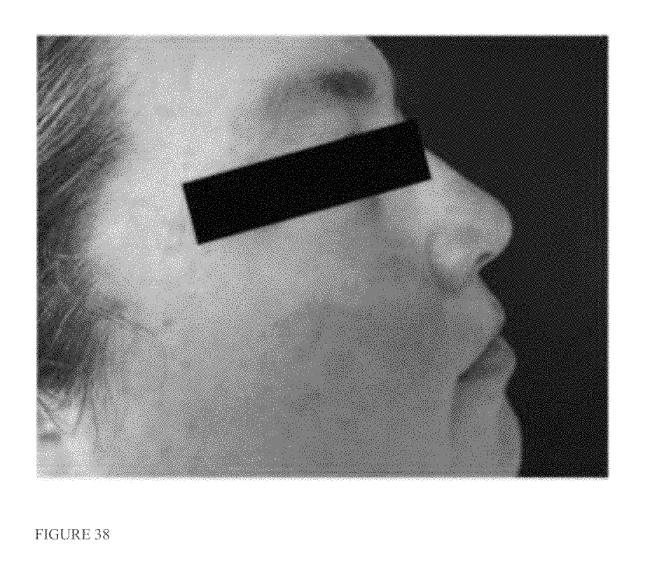

FIGS. 37 and 38 show color photographs of the patient's face after two months of treatment. These photographs demonstrate almost complete amelioration of inflammation and discoloration. No adverse side effects were observed.

### Example 11

A topical gel is made with delphinidin 3,5-O-diglucoside 7% (w/w) in a standard cosmetic disappearing gel base. Application b.i.d. to the face of a patient with lupus-related rosacea, for at least about one month alleviates lupus-related skin inflammation and the patient maintains normal, healthy skin.

### Example 12

A topical cream is made with delphinidin 6% (w/w) in a standard cosmetic lipophilic cream base. Application b.i.d. to the face of a patient with erythematotelangiectatic rosacea featuring telangiectasia for two weeks measurably reduces flushing, persistent central facial erythema and telangiectasia.

### Example 13

A topical cream is made with delphinidin 3-O-sambubioside 5-O-glucoside 3% (w/w) in a standard cosmetic disappearing cream base. Application b.i.d. to the face of a patient with papulopustular rosacea with transient, central face pustules for two months reduces central facial erythema and eliminates transient central face pustules.

### SUMMARY

For clarity, the appended legal claims use the term “delphinidin” (lower case “d”) to refer to 2-(3,4,5-trihydroxyphenyl) chromenylium-3,5,7-triol (CAS 13270-61-1), and use the term “Delphinidin” (with a capital “D”) to refer to CAS 13270-61-1 and to structurally-similar compounds which provide a functionally-similar benefit, e.g., delphinidin 3-O-sambubioside 5-O-glucoside, delphinidin 3,5-O-diglucoside, delphinidin 3-O-sambubioside and delphinidin 3-O-glucoside.

We claim:

1. A topical cream comprising Delphinidin in an amount effective to ameliorate dermal inflammation due to rosacea or lupus.

2. The topical cream of claim 1, comprising at least about 1% (w/w) Delphinidin.

3. The topical cream of claim 2, comprising at least about 10% (w/w) Delphinidin.

4. The topical cream of claim 1, wherein the Delphinidin comprises at least one glucoside moiety.

5. The topical cream of claim 4, wherein the Delphinidin comprises at least one compound selected from the group consisting of: delphinidin-3-O-sambubioside 5-O-glucoside, delphinidin 3,5-O-diglucoside, delphinidin-3-O-sambubioside and delphinidin-3-O-glucoside.

6. The method of claim 5, wherein the Delphinidin comprises delphinidin-3-O-sambubioside 5-O-glucoside, delphinidin 3,5-O-diglucoside, delphinidin-3-O-sambubioside and delphinidin-3-O-glucoside.

7. A method comprising: applying the topical cream of claim 1 to the skin of a human with a condition selected from the group consisting of rosacea and lupus.

8. A method comprising: applying the topical cream of claim 2 to the skin of a human with a condition selected from the group consisting of rosacea and lupus.

9. A method comprising: applying the topical cream of claim 3 to the skin of a human with a condition selected from the group consisting of rosacea and lupus.

10. The method of claim 7, where the condition comprises rosacea.

11. The method of claim 7, where the condition comprises lupus.
